(19) United States
(12) Patent Application Publication
(10) Pub. No.: US 2024/0191260 A1
(43) Pub. Date: Jun. 13, 2024

(54) METHODS OF PREPARING STABLE NUCLEIC ACID LIQUID FORMULATIONS

(71) Applicant: University of North Carolina at Wilmington, Wilmington, NC (US)

(72) Inventors: Ying Wang, Kure Beach, NC (US); Harrison R. Wooten, Wilmington, NC (US); Carson Jackson, Wilmington, NC (US)

(73) Assignee: University of North Carolina at Wilmington, Wilmington, NC (US)

(21) Appl. No.: 18/534,290

(22) Filed: Dec. 8, 2023

Related U.S. Application Data
(60) Provisional application No. 63/431,167, filed on Dec. 8, 2022.

(51) Int. Cl. C12N 15/88; A61K 47/69; A61K 48/00

(52) U.S. Cl. CPC C12N 15/88; A61K 47/6933; A61K 47/6935; A61K 48/0041

(57) ABSTRACT

Stable nucleic acid liquid formulations and methods of making and using the same are provided. According to some aspects, a method for preparing stable nucleic acid liquid formulations are provided that are resistant to nucleic acid degradation caused by enzyme-independent or enzymatic hydrolysis. In some instances, stable nucleic acid liquid compositions and methods or making the same incorporate the use of at least one of a polymer and/or a salt to form a macroscopic RNA-rich condensate. In other aspects, a method for preparing stable nucleic acid encapsulated lipid nanoparticle (LNP) liquid formulations is provided that are resistant to degradation caused by nucleic acid hydrolysis, LNP leakage, and LNP aggregation. In some instances, stable nucleic acid liquid compositions and methods or making the same incorporate the use of a pair of thermodynamically matching LNP synthesis buffer and LNP product formulation buffer with close-to-equal osmotic pressure and chemical potentials of solution components.

### CROSS-REFERENCE TO RELATED APPLICATIONS

This application claims priority to U.S. Provisional Patent Application No. 63/431,167 filed Dec. 8, 2022, which is incorporated herein by reference in its entirety.

### FIELD

The technology described herein generally relates to nucleic acid liquid formulations, and more particularly to preparing and/or forming stable nucleic acid liquid formulations using polymer-induced nucleic acid condensation, and further relating to nucleic acid encapsulated lipid nanoparticle liquid formulations.

### BACKGROUND

Nucleic acids including, but not limited to, deoxyribonucleic acids (DNAs), and ribonucleic acids (e.g. mRNAs, siRNAs, and tRNAs) are widely used as therapeutics or research reagents. Nucleic acids undergo hydrolysis degradation in aqueous solutions which can be exacerbated by elevation of temperature or the contamination of DNases and RNases. Therefore, the nucleic acid liquid formulations often require ultra-cold storage conditions and further have limited shelf life when they are not frozen.

Furthermore, pharmaceutical nucleic acids are usually encapsulated in lipid nanoparticles (LNPs) for DNA and RNA vaccines and therapeutics. As will be appreciated, nucleic acid encapsulated LNPs are also prone to degradation in aqueous solutions through various mechanisms, such as LNP aggregation, hydrolysis of nucleic acids, nucleic acid leakage from LNPs, and oxidation. In order to mitigate these degradation mechanisms, nucleic acid encapsulated LNP solutions can be kept frozen or in form of lyophilized powders. However, drying and freezing processes are known to cause aggregation of LNPs which in turn leads to loss of biological activity of DNA-LNPs or RNA-LNPs. On the other hand, liquid formulations of nucleic acid encapsulated LNPs usually have limited shelf life when they are not frozen.

Consequently, there is a need to provide preparations of improved solutions of nucleic acid drug substances or nucleic acid solution samples, for instance for pharmaceutical and/or research use, that can be stored, transported, and used at refrigerated or ambient temperatures.

The solutions or formulations provided by the present technology work to eliminate the cold-chain requirements for transporting and storing nucleic acid solutions, decrease the costs of distribution, and lower the risk of wastes due to potential exposure to elevated temperature during transportation and/or storage and contamination by DNases/RNases.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Embodiments of the technology described herein are directed towards methods for preparing or producing stable liquid formulations of nucleic acids, for instance for pharmaceutical and/or research use. In some instances, methods and compositions are provided herein to form RNA-rich and/or DNA-rich condensates (e.g., droplets or granules) through macroscopic liquid-liquid phase separation. In some instances, condensates provided herein may be induced with cationic polymers.

According to some embodiments, a method for preparing a stable nucleic acid liquid formulation comprises providing an RNA and/or DNA solution having an initial nucleic acid (e.g. DNA, RNA) concentration, adding at least one polymer, adding at least one salt, and forming a macroscopic RNA-rich and/or DNA-rich condensate, in some instances having morphologies such as droplets or granules. The polymer may be at least one of a biocompatible crowding polymer and a cationic polymer. In some instances, the polymer can be at least one of polyethylene glycol (PEG) and polyvinyl pyrrolidone. In some other instances, the salt can be sodium chloride (NaCl). In some instances, at least 95% of the RNA or DNA in solution is partitioned into the RNA-rich and/or DNA-rich condensate. Further, the method comprises dissolving the RNA-rich and/or DNA-rich condensate to return the nucleic acid concentration to the initial concentration. According to some aspects, the formed RNA-rich and/or DNA-rich condensate does not undergo any significant degradation of the nucleic acid for at least a week at a temperature of about 20° C.

According to some further embodiments, a composition of buffers and excipients is provided for enhancing the thermostability of RNA-rich condensates formed in the presence of such formulation components. In some aspects, examples of the RNA stability enhancing buffers and excipients can include citrate, succinate, tris (pH between 8 and 9), bis-tris, 1-2-3-4-Butanetetracarboxylic Acid, tryptophan, glutamic acid, arginine, proline, phenylalanine, poloxamer, tween, and/or cyclodextrin. In contrast, a composition of other buffers and excipients is provided where the RNA or DNA-rich condensates formed in the presence of these formulation components have decreased thermostability and should be avoided in the preparation of RNA condensate formulations. Examples of the RNA destabilizing buffers and excipients include acetate, histidine, phosphate, tris at pH<8, lysine.

According to some further embodiments compositions of pairs of formulation buffers are provided for preparing DNA or RNA encapsulated LNPs with enhanced thermostability. Each pair of formulation buffers consist of at least one acidic buffer for LNP synthesis and one near-neutral buffer for the final LNP product formulation. The LNP synthesis buffer contains at least one RNA stabilizing buffer salt, for example, citrate or 1-2-3-4-butanetetracarboxylic acid, at pH between 3 and 4 (and cannot contain acetate) and at least one other RNA stabilizing excipients, for example, phenylalanine arginine, glutamic acid, glycine, proline, tween, poloxamer, tween, and cyclodextrin. The LNP product formulation buffer contains at least one RNA stabilizing buffer such as tris, bis-Tris, citrate, or 1-2-3-4-butanetetracarboxylic acid at pH between 6 and 8.5 (cannot contain phosphate and histidine), at least one crowding reagent such as PEG, and at least one other RNA stabilizing excipients, for example, phenylalanine arginine, glutamic acid, glycine, proline, tween, poloxamer, tween, and cyclodextrin. The concentrations of the excipients in the LNP synthesis buffer and the LNP product formulation buffer were determined in the range from 0 mM to 500 mM to achieve close-to-equal osmotic pressures and chemical potentials in the two buffers. One example method to match the osmotic pressures and chemical potentials of the final product formulation buffer to the LNP synthesis buffer is to place the two buffers with various concentration of excipients in a container separated by a semi-permeable membrane. The excipient concentrations in the two buffers result in least and slowest buffer exchange across the membrane are the most viable excipient concentrations for the two thermodynamically matching buffers.

According to some even further embodiments, a kit for thermostable DNA/RNA condensate formulation is provided, the kit comprising a buffer, a polymer salt mix, and a diluent.

According to some even further embodiments, a kit for thermostable DNA/RNA-LNP formulation is provided, the kit comprising a LNP synthesis buffer, a LNP dialysis buffer, and an excipient mix.

Additional objects, advantages, and novel features of the invention will be set forth in part in the description which follows, and in part will become apparent to those skilled in the art upon examination of the following, or can be learned by practice of the invention.

### BRIEF DESCRIPTION OF THE DRAWINGS

Aspects of the technology presented herein are described in detail below with reference to the accompanying drawing figures, wherein:

FIG. 1A illustrates cytoplasmic RNP granules in a cell and RNAs and proteins in a RNP granule;

FIG. 1B shows RNA-rich condensates of PolyA without proteins, in accordance with some aspects of the technology described herein;

FIG. 1C shows RNA-rich condensates of GFP mRNA without proteins, in accordance with some aspects of the technology described herein;

FIG. 2 illustrates the efficient formation and dissolution of RNA condensates, in accordance with some aspects of the technology described herein;

FIG. 3 illustrates RNA degradation during incubation at a temperature of 21° C., in accordance with some aspects of the technology described herein;

FIG. 4 illustrates condensates in polyA RNA solutions under a light microscope, in accordance with some aspects of the technology described herein;

FIG. 5 illustrates condensates for mRNAs, in accordance with some aspects of the technology described herein;

FIG. 6A illustrates concentration of Poly-A in supernatant after encapsulation in condensate at 21° C., in accordance with some aspects of the technology described herein;

FIG. 6B illustrates concentration of GFP mRNA in supernatant after encapsulation in condensate at 21° C., in accordance with some aspects of the technology described herein;

FIG. 7A illustrates the dissolution of Poly-A in a condensate, in accordance with some aspects of the technology described herein;

FIG. 7B illustrates the dissolution of GFP mRNA in a condensate, in accordance with some aspects of the technology described herein;

FIG. 8 illustrates condensate formation at varying polymer concentration (6% w/w PEG3350) induced in phosphate buffered saline, in accordance with some aspects of the technology described herein;

FIG. 9 illustrates condensate formation at varying polymer concentration (8% w/w PEG3350) induced in phosphate buffered saline, in accordance with some aspects of the technology described herein;

FIG. 10 illustrates condensate formation at varying polymer concentration (10% w/w PEG3350) induced in phosphate buffered saline, in accordance with some aspects of the technology described herein;

FIG. 11A illustrates concentrations of a GFP mRNA in supernatants after encapsulation in condensates at various PEG concentrations, in accordance with some aspects of the technology described herein;

FIG. 11B illustrates dissolution of a GFP mRNA condensates, in accordance with some aspects of the technology described herein;

FIG. 12A illustrates the effects of solution salinity on RNA condensate formation at 0.18 M NaCl, in accordance with some aspects of the technology described herein;

FIG. 12B illustrates the effects of solution salinity on RNA condensate formation at 0.35 M NaCl, in accordance with some aspects of the technology described herein;

FIG. 12C illustrates the effects of solution salinity on RNA condensate formation at 0.4 M NaCl, in accordance with some aspects of the technology described herein;

FIG. 12D illustrates the effects of solution salinity on RNA condensate formation at 0.5M NaCl, in accordance with some aspects of the technology described herein;

FIG. 12E illustrates the effects of solution salinity on RNA condensate formation at 0.58M NaCl, in accordance with some aspects of the technology described herein;

FIG. 13A illustrates RNA condensate formation in the presence of cationic polymers, in accordance with some aspects of the technology described herein;

FIG. 13B illustrates RNA condensate formation in the presence of cationic polymers, in accordance with some aspects of the technology described herein;

FIG. 13C illustrates RNA condensate formation in the presence of cationic polymers, in accordance with some aspects of the technology described herein;

FIG. 14 illustrates a GFP mRNA degradation in citrate buffer, in accordance with some aspects of the technology described herein;

FIG. 15A illustrates two thermodynamically matching buffers used in mRNA encapsulated production, in accordance with some aspects of the technology described herein;

FIG. 15B illustrates two thermodynamically matching buffers used in mRNA encapsulated production, in accordance with some aspects of the technology described herein; and

FIG. 16 illustrates electrophoresis results of a GFP mRNA stability in granule formulations in various formulation, in accordance with some aspects of the technology described herein.

### DETAILED DESCRIPTION

The subject matter of aspects of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps disclosed herein unless and except when the order of individual steps is explicitly described.

Accordingly, embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

Additionally, in any disclosed embodiment, the terms “substantially,” “approximately,” and “about” may be substituted with “within [a percentage] of” what is specified, where the percentage includes 0.1, 1, 5, and 10 percent.

Ribonucleic acids (RNA), e.g., mRNA and siRNA, are an emerging family of biologics used for treatment of cancers, hereditary disorders, and infectious diseases. RNA pharmaceuticals offer safety and efficacy superior to traditional drugs and vaccines. However, distribution and storage of RNA pharmaceuticals are impeded by rapid degradation of RNA molecules in aqueous solutions. Furthermore, RNA vaccines and drugs are usually formulated in encapsulation by lipid nanoparticles (LNP) which introduces a new mechanism of RNA degradation induced by oxidized lipids. Accordingly, both mRNA-LNP and naked mRNA solutions require ultra-cold chain transportation and storage, which significantly increase the logistics costs, drug/vaccine wastage, and medical inequality.

RNA degradation in aqueous solutions is mediated by hydrolysis of the backbone diester bonds and oxidation of nitrogenous bases. Oxidation of RNA without high level of free radicals is typically not a concern for storage due to the long half-life of oxidation of bases (e.g., >38 years for 8-oxoguanine, the major oxidation product, formation at 37° C.). On the other hand, the rate of RNA hydrolysis strongly depends on pH, temperature, cation concentration, and the secondary structure of RNA. Estimated by the spontaneous hydrolysis rate of RNAs under physiological conditions, the half-life of an mRNA with ˜4000 nucleotides would be merely ˜20 hours. In real-world situations or applications, RNA hydrolysis can be greatly accelerated by the contamination of ribonuclease (RNase) enzymes which is found ubiquitously in the environment. As will be appreciated, therefore, RNA's in aqueous formulations mainly degrade through increased or fast hydrolysis during storage and/or transportation.

To mitigate RNA hydrolysis, RNA solutions are often frozen or dried (lyophilized). Freezing and lyophilization can efficiently slow down RNA degradation, but both methods have the disadvantages of additional costs, time-consuming, and limited capacity. For the mRNA-LNP formulations, freezing and lyophilization could also cause aggregation of LNP and loss of mRNA-LNP efficacy. Furthermore, the dried powders of RNA are prone to absorption of moistures from air and require humidity controlled storage conditions. According to some known methods, air- and water-tight stainless steel mini-capsules have been utilized to store RNAs in an anhydrous and anoxic environment. Kinetic studies at 90° C.(equivalent to 160 years at room temperature) showed that dried RNA in these stainless steel mini-capsules did not exhibit significant degradation. Another conventional approach to increasing RNA stability is through designing secondary structure of RNA molecules. Studies have shown that a two-fold increase of mRNA in vitro half-life can be achieved by optimizing a given RNA sequence to form stabilizing secondary structures. However, this approach is limited by the design space of the RNA sequencing and the risk of compromising pharmaceutical efficacy of the base structures.

At a high level, embodiments of the technology described herein are directed towards nucleic acid liquid formulations, and more particularly, to preparing and/or forming stable nucleic acid liquid formulations using polymer-induced nucleic acid condensation, for example in one instance the formation of droplets or granules enriched with nucleic acids. In some other embodiments, the technology described herein is directed towards nucleic acid encapsulated lipid nanoparticle liquid formulations, and more particularly to preparing and/or forming stable nucleic acid encapsulated lipid nanoparticle liquid formulations using two matching buffers in thermodynamic equilibrium for lipid nanoparticles.

Accordingly, in some aspects methods described herein are directed to preparing and/or forming stable nucleic acid liquid formulations that are resistant to nucleic acid degradation caused by elevated temperature or enzymatic digestion (DNAase or RNase). Further, methods and/or formulations or compositions are provided for preparing or prepared stable liquid formulations of RNA with or without lipid vesicles or lipid nanoparticles (LNP) which may be utilized in a variety of applications, for instance pharmaceutical or research use. As will be appreciated RNAs (including but not limited to DNAs, mRNAs, siRNAs, and tRNAs) are widely used as therapeutics or research reagents. RNAs undergo hydrolysis degradation in aqueous solutions which can be exacerbated by elevation of temperature or the contamination of RNases. Furthermore, LNPs are prone to aggregation and leakage of encapsulated nucleic acids, which leads to loss of the pharmaceutical efficacy of RNA-LNP. Therefore, conventional RNA-LNP based pharmaceuticals often need to be frozen or dried for storage and transportation.

According to various aspects, methods and compositions and/or formulations are provided for stable nucleic acid liquid formulations utilizing polymer-induced nucleic acid condensation. The methods or preparation and the formulations or compositions as descried herein can provide multiple advantages over conventional methods or formulations. At a high level, and without limitation, the technology described herein can include the preparation of RNA condensates using salt(s) and/or polymer polyethylene glycol (PEG). Further, according to aspects of the present technology, thermostable RNA condensates can be prepared under different solution conditions (i.e. versatile formulation conditions) that can include different buffers (e.g., citrate, succinate, tris (pH between 8 and 9), bis-tris, 1-2-3-4-butanetetracarboxylic acid) at different pH, different other excipients (tryptophan, glutamic acid, arginine, proline, phenylalanine, poloxamer, tween, cyclodextrin), a broad range of solution salinity (or salt concentration), various RNA concentrations and PEG concentrations, as well as with or without different cationic polymers. According to some other aspects, RNA condensates prepared with some other buffers and excipients, e.g., acetate, histidine, phosphate, tris at pH<8, and lysine, have low thermostability. Even further, according to some aspects, RNA can be encapsulated efficiently (e.g. >95%) in these condensates and can be fully recovered prior to us by, for instance, dilution and/or heating, which are generally known to be important aspects for applications in pharmaceutical formulations. According to some even further aspects, the RNA degradation according to the present technology is significantly slower in the condensates as compared to conventional solutions without condensates when stored at ambient temperatures (e.g. room temperature). It will be appreciated that condensate, as referred to herein, generally can refer to the macroscopic nucleic acid-rich high-density entities that are visible under light microscope with the morphologies of droplets, granules, or aggregates.

According to embodiments of the technology described herein, improved methods for preparing, forming, and/or producing stable liquid formulations of nucleic acids, including RNA and DNA are provided. According to some aspects, methods and/or formulations as described herein can use biocompatible crowding polymers (e.g. polyethylene glycol) and/or cationic polymers (e.g. polyvinyl pyrrolidone) to induce formation of DNA or RNA-rich condensates in the liquid formulations. Liquid formulations using and/or containing these nucleic acid condensates are stable for a prolonged time period without being frozen. Further, these nucleic acids in the condensates are also significantly resistant to degradation by high level of DNase or RNase. The condensates are fully dissolved upon dilution or heating to release active nucleic acids.

According to some aspects, the method and/or formulations described herein can be utilized to prepare or produce, for example, liquid nucleic acid drug products, raw nucleic acid materials in solutions, and/or nucleic acid liquid samples that can be stored, transported, and used at refrigerated or ambient temperatures.

In some further embodiments, methods of preparing stable liquid formulations of nucleic acid encapsulated lipid nanoparticles (DNA/RNA-LNP) are provided. According to some aspects, one acidic buffer can be used for synthesizing a pharmaceutical DNA/RNA-LNP active ingredient. The LNP synthesis buffer contains at least one RNA stabilizing buffer salt, for example, citrate or 1-2-3-4-butanetetracarboxylic acid, at pH between 3 and 4 (and cannot contain acetate) and at least one other RNA stabilizing excipients, for example, phenylalanine arginine, glutamic acid, glycine, proline, tween, poloxamer, tween, and cyclodextrin. According to some other aspects, the synthesized DNA/RNA-LNP ingredients are dialyzed into another LNP product formulation buffer that contains at least one RNA stabilizing buffer, such as tris, bis-Tris, citrate, or 1-2-3-4-butanetetracarboxylic acid at pH between 6 and 8.5 (and cannot contain phosphate and histidine), at least one crowding reagent, such as PEG, and at least one other RNA stabilizing excipient, for example, phenylalanine arginine, glutamic acid, glycine, proline, tween, poloxamer, tween, and cyclodextrin. The concentrations of the excipients in the LNP synthesis buffer in the presence of nucleic acid cargo and the LNP product formulation buffer containing macromolecular crowding agents in absence of nucleic acid cargo in some instances are determined to be in the range from 0 mM to 500 mM to achieve close-to-equal osmotic pressures and chemical potentials in the two buffers. In some aspects, adding a macromolecular crowding reagent and at least one nucleic acid stabilizing excipient at the series of concentrations to the DNA/LNP-LNP formulation after buffer exchange to achieve osmotic pressure and chemical potentials close-to-equal to the LNP synthesis buffer.

As will be appreciated, and as described herein, the methods and formulations of the present technology can provide liquid DNA/RNA-LNP compositions (e.g. as or for pharmaceutical products), that can be transported and/or stored without the need for freezing or drying (e.g. lyophilization). Accordingly, liquid formulations having improved stability properties in accordance with the technology described herein can decrease distribution costs and lower the risks of waste associated with exposure to elevated temperatures (i.e. above freezing point) for example during transportation and/or storage.

In some aspects, the macrocrowding reagent including polyethylene glycol (PEG) used differs from conjugated PEG's that are covalently connected to LNPs. The conjugated PEG's are integrated as a part of an LNP, while the PEG utilized in the present formulations are external free excipients added into mRNA-LNP solutions or compositions. The free PEG excipients (i.e. additives) can be used at various different concentrations and various different molecular weights, while conjugated-PEG has a fixed percentage and molecular weight in a given LNP composition. Without intending to be bound by theory, the free PEG additives can significantly increase the thermostability of mRNA-LNPs that cannot be achieved by PEG conjugation of LNP alone. Further, RNA hydrolysis degradation is known to be slower at lower pH. However, in accordance with embodiments of the present technology, RNA-LNP can have improved thermostability in acidic buffers. As will be appreciated, because RNA-LNPs are integrated complexes, it has not been previously demonstrated that RNA-LNPs would be more stable in moderately acidic buffers, e.g., citrate buffer from pH 4.5 to pH 6.5. Accordingly, the present technology utilizes a combination of macromolecular crowding agents and moderately acidic to acidic buffers to address at least two mechanisms of degradation of RNA-LNP complexes in solutions, i.e., aggregation of RNA-LNP and hydrolysis of RNA.

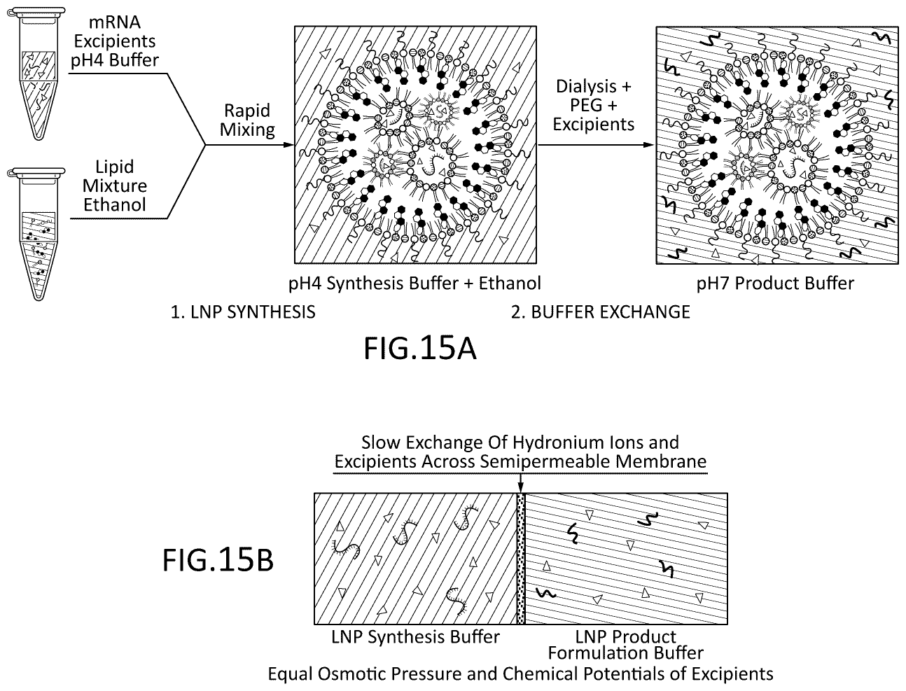

In some aspects, a pair of buffers can be used to produce DNA/RNA encapsulated LNPs with enhanced thermostability DNA/RNA-LNP manufacturing comprises at least two steps using two different buffers. In one example, an acidic buffer at pH 3 or 4 is used for DNA/RNA-LNPs synthesis. After synthesis, buffer exchange through dialysis is performed to prepare the final LNP product formulation in a near-neutral buffer. Previously, citrate and acetate buffers at pH between 3 and 4 without other excipients have been used for synthesis of DNA/RNA encapsulated LNPs. Referring to FIG. 15A, a scheme of RNA-LNP production using a pair of matching formulations is provided. FIGS. 15A and 15B shows two thermodynamically matching buffers that are used in two steps of mRNA production, that is for example a pH 4 buffer for LNP synthesis by rapid mixing and a pH 7 buffer for buffer exchange by dialysis. In some aspects, a synthesis buffer containing excipients and a matching product buffer containing matching excipients and a macromolecular crowding agent (e.g. PEG).

As illustrated in FIGS. 15A and 15B, a formation buffer for DNA/RNA-LNP synthesis comprises utilizing a citrate buffer or 1-2-3-4-butanetetracarboxylic acid at pH between 3 and 4 and at least on nucleic acid stabilizing excipient, for example, tryptophan, phenylalanine, tyrosine, arginine, glutamic acid, glycine, proline, tween, poloxamer, tween, and cyclodextrin, at the concentration ranging from 0 to 500 mM. It will be appreciated that acetate buffer, histidine, and lysine accelerate nucleic acid hydrolysis degradation and should be avoided. After DNA/RNA-LNP synthesis, a formulation for buffer exchange can use at least one RNA stabilizing buffer, such as tris, bis-Tris, citrate, or 1-2-3-4-butanetetracarboxylic acid at pH between 6 and 8.5, and at least one other RNA stabilizing excipient(s), for example, phenylalanine arginine, glutamic acid, glycine, proline, tween, poloxamer, tween, and cyclodextrin. After buffer exchange, at least one crowding reagent or agent such as PEG is added to the final DNA/RNA-LNP product formulation. In some instances, the DNA/RNA-LNP synthesis formulation and the DNA/RNA-LNP final product formulation can be designed and used in pair to produce thermostable DNA/RNA-LNP liquid formulations. The concentrations of the excipients in the LNP synthesis buffer and the LNP product formulation buffer can be determined to be in the range from 0 mM to 500 mM to achieve close-to-equal osmotic pressures and chemical potentials (near thermodynamic equilibrium) in the two buffers. To determine good excipient concentrations for the two thermodynamically matching buffers, the LNP synthesis buffer and the final product formulation buffer with various concentrations of excipients described above are placed in a container separated by a semi-permeable membrane. The hydronium ion and excipient concentrations in the two buffers result in the least and slowest buffer exchange across the membrane are used to prepare the two matching buffers for thermostable RNA-LNP production. Through achieving the near thermodynamic equilibrium state, the matching pairs of acidic DNA/RNA-LNP synthesis formulation and near-neutral DNA/RNA-LNP product formulation are utilized to mitigate at least three mechanisms of degradation of DNA/RNA-LNP complexes in solutions, i.e., aggregation of LNP, hydrolysis of nucleic acids, and leakage of the encapsulated nucleic acids from LNPs. As will be appreciated, FIG. 15B further illustrates the mechanisms comprising a slow exchange of hydronium ions and excipients across a membrane, for example a semi-permeable membrane. In this way equal osmotic pressure and chemical potentials of excipients can be achieved.

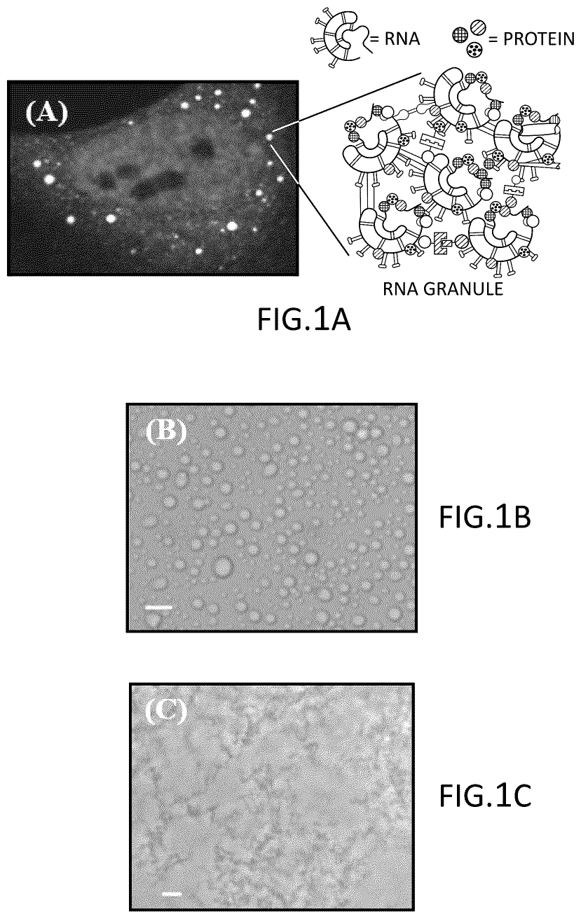

According to some aspects, the present technology is directed towards stable liquid RNA formulations, which in effect slow down RNA hydrolysis. In other words, the methods, compositions and/or formulations described herein can reduce the mobility of RNA molecules and the water content in an aqueous solution. In order to achieve these formulations, the present technology leverages a natural “RNA-warehouse” in cells. As will be appreciated, and with reference to FIG. 1A, eukaryote cells have cytoplasmic membrane-less organelles in some instances known as ribonucleoprotein granules (RNPs). These RNPs are droplet- or gel-like condensates packed with RNAs and numerous RNA-binding proteins. As shown in FIG. 1A, cytoplasmic RNP granules are shown in a cell, and an illustration of RNAs and proteins in an RNP granule are also shown. As can be appreciated FIG. 1 illustrates cytoplasmic RNP granules in a cell and RNAs and proteins in a RNP granule.

With reference to FIGS. 1B and 1C, methods are provided to create or generate in vitro RNA-rich droplets like RNPs without proteins through liquid-liquid phase separation (LLPS), as will be appreciated, the white scale bar is 10 μm. Accordingly, the formation of the mRNA-rich droplets can be induced by adding or introducing polymers (e.g., PEG) and salts (e.g., NaCl) into RNA solutions. As will be appreciated, these RNA-rich droplets are stable at room temperature for an extended period of time (e.g. more than 3 months), and even exist for a week or more in the presence high concentration of RNase which normally degrades all RNAs within a few minutes. Looking at FIG. 1B, RNA-rich condensates (large droplets) of PolyA without proteins at 20° C. made in accordance with the present technology are depicted, and looking at FIG. 1C, RNA-rich condensates (small granules) of GFP mRNA without proteins at 20° C. made in accordance with the present technology are depicted. As will be appreciated, the white bars in FIGS. 1B and 1C represent 10 μm.

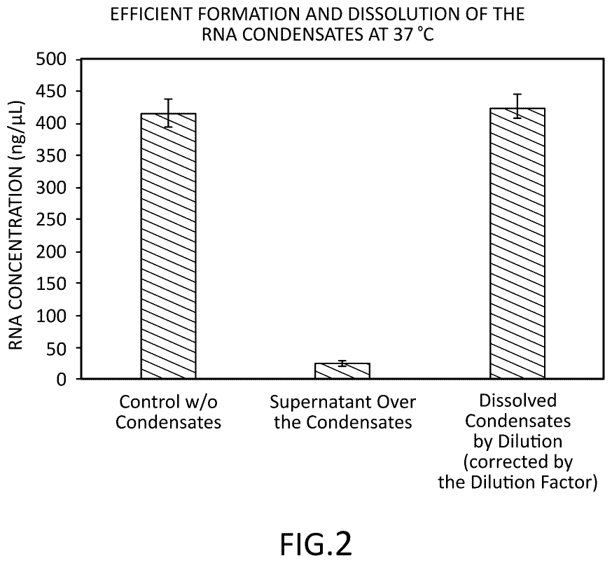

Further, according to some aspects, it was determined how much RNA partitioned into the RNA-rich condensates. Referring to FIG. 2, it is shown that less than 5% RNA remains in the supernatant after spinning down of condensates, i.e., >95% RNA is packed in the condensates. Furthermore, the condensates were completely dissolved upon 2× dilution with water. Accordingly, as illustrated, RNA concentration after dissolving the condensates was restored to its original value (after corrected by the dilution factor). As will be appreciated, high partitioning of RNA into the condensates and complete dissolution of the condensates upon dilution are important for developing practically useful RNA formulations. As shown in FIG. 2, RNA concentrations of a uniform RNA solution without condensates (left), the supernatants over the condensates formed in a solution with the same total RNA concentration (middle), and the dissolved condensates by 2× dilution with water (right).

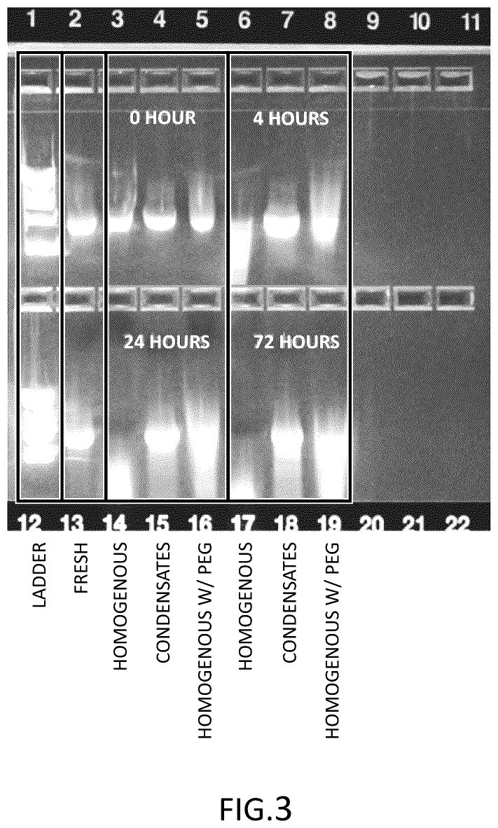

According to some even further aspects, sample formulations were used to run agarose gel electrophoresis to examine RNA degradation during incubation at room temperature as illustrated in FIG. 3. As depicted, three types of samples including homogenous mRNA solutions in pH 6 citrate buffer with NaCl, formulations containing mRNA condensates in the same buffer with PEG and NaCl, and homogenous mRNA solutions with PEG but without NaCl. The samples were incubated at room temperature)(21° C. for different times up to three days. With reference to FIG. 3, it is shown that that no significant degradation of mRNA in the condensates at room temperature for at least three days. Without intending to be bound by theory, this stability may be attributed to the low mobility and low water content in the gel-like condensates. As shown in FIG. 3, the aging of GFP mRNA samples at 20° C. is depicted. From the left to right, the samples are (1) RNA ladder; (2) fresh mRNA; (3, red) homogenous mRNA solution in pH 6 citrate buffer with NaCl; (4, green) emulsion with mRNA condensates in the same buffer with PEG and NaCl, (5, orange) homogenous mRNA solution in the same buffer with PEG without NaCl.

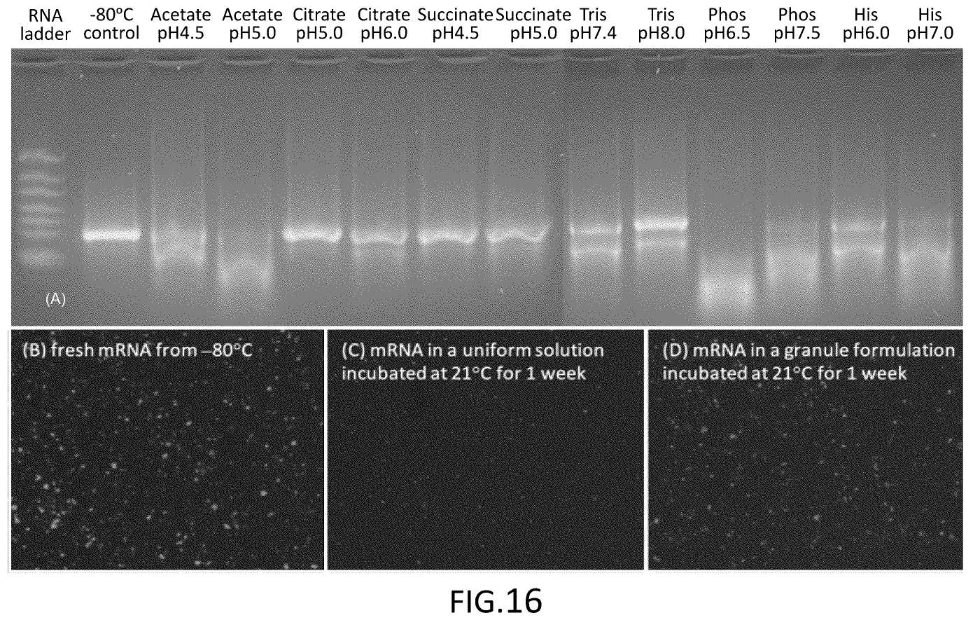

According to some further aspects, RNA condensate formulations were prepared in 12 buffers including sodium acetate at pH 4.5 and 5.0, sodium succinate at pH 4.5 and 5.0, sodium citrate at pH 5.0 and 6.0, histidine at pH 6.0 and pH 7.0, sodium phosphate at pH 6.5 and 7.5, tris hydrochloride at pH 7.4 and 8.0. After 7-day incubation at 21° C., the RNA condensates were dissolved with 5 times volume of nuclease-free water and heated at 37ºC for 15 minutes. Then, agarose gel electrophoresis was used to check the mRNA integrity as the results shown in FIG. 16. Referring to FIG. 16, the integrity and in vitro transfection activity of TriLink eGFP mRNA in various granule formulations after incubation at 21° C. for one week is illustrated. FIG. 16A shows gel electrophoresis results of various granule formulations, and FIGS. 16B-D show fluorescence from A549 cells transfected with GFP mRNA from three different conditions.

Accordingly, FIG. 16 illustrates electrophoresis results of a GFP mRNA stability after incubation at 21° C. for 1 week in granule formulations in various formulation buffers, as well as illustrates GFP expression in A549 cell cultures using the mRNA after incubation. FIG. 16 shows that the mRNA in the condensates formulations prepared with citrate pH 5.0, succinate pH 4.5 and 5.0, and tris pH 8.0 and excipients remain intact after incubation at 21° C., while those from the condensates prepared in the other buffers are highly degraded. In addition, the activity of the mRNA in the condensate formulations were tested by in vitro transfection of A549 cell cultures and measured using a green fluorescence imaging system (FIG. 16 B,C,D) The optimal mRNA granule formulations retained >95% in vitro transfection activity (FIG. 16 B,C,D) after 1-week storage at room temperature.

According to some other embodiments of the present technology, thermostable DNA/RNA condensate formulation buffer kits and methods of preparation are provided. In some instances, a kit can include one or more reagents, for instance a condensate formulation kit can include a buffer, a polymer salt mix, and a diluent. In one example, a DNA/RNA condensate formulation kit or buffer kit can include an (A1) buffer, an (A2) polymer salt mix, and a (B1) diluent. In some aspects, a buffer can include a concentrated buffer consisting of at least one buffer including citrate (pH between 3 and 6.5) or 1-2-3-4-butanetetracarboxylic acid (pH between 3 and 6.5) or tris (pH between 6.0 and 8.5) or bis-Tris (pH between 6.0 and 8.5) at buffer concentration 40 mM to 1 M, and one or more excipients including phenylalanine arginine, glutamic acid, glycine, proline, tween, poloxamer, tween, and cyclodextrin at concentration from 0 to 500 mM. A polymer salt mix can include a solution of NaCl and polymer, e.g., polyethylene glycol (PEG) with molecular weight 3350 (PEG3350) at 0-50% weight percentage. PEG with other molecular weights can also be used. NaCl or other salt (KCl) at concentration from 0 to 2M. A diluent can include or consist of nuclease-free water or pH 6.5 citrate (10 500 mM) or pH 8 tris (10-500 mM).

In some further embodiments, a method of preparation of thermostable DNA/RNA condensate formulations is provided. In one example, with reference to the above described kit, a protocol can include first mixing DNA/RNA samples with initial concentration, C_{XNA.0}, equal to or larger than 1 μg/μL and a first reagent (e.g. reagent A1) at the volume ratio VANA:1/1=2:1 in a 1.5-mL nuclease-free microcentrifuge tube (the tube should be able to hold up to 12 times of VANA solutions), e.g., mix 100 μL 1 mg/mL RNA solution with 50 μL of the first reagent (e.g. reagent A1). Mixing in some instances can be carried out by tapping the tube or repeated pipetting. Subsequently, a second reagent (e.g. reagent A2) is added at the same amount as the first reagent (e.g. reagent A1) to the same tube, where V₂=V₁, e.g., 50 μL of the second reagent (e.g. reagent A2). The reagents can be mixed thoroughly, for example by repeated pipetting. Finally, the mixed solution can be stored at ambient temperature (e.g. ˜ 20° C.)or in a refrigerator at, for example 4-8° C. The formulation can be recorded as volume per tube V=2V_{XNA} (or 4V₁), e.g., 200 μL, and DNA/RNA concentration C_{XNA}-C_{XNA.0}/2, e.g., 500 ng/μL. It would be recommended to label V and C_{XNA} on the tube.

As will be appreciated, the volume ratio of DNA/RNA solutions, reagent 1, and reagent 2 V_{DNA RNA}:V₁: V₂=2:1:1 is an example. Other volume ratios can also be used with the concentration of reagents adjusted accordingly. Further, if aliquots need to be made for storage, DNA/RNA solutions should be aliquoted before preparing condensate formulation. Aliquoting DNA/RNA condensate formulations is not recommended because transferring exact amount of condensates is difficult.

In some further embodiments, methods of using DNA/RNA condensates are provided. In some instances, the DNA/RNA condensates can be dissolved before use. Accordingly, the dissolution of thermostable DNA/RNA condensate formulations prior to use can be carried out. The condensate formulation excipients are compatible with most applications of DNAs and RNAs. If needed, these excipients (including high concentration of PEG) can be removed in this step. A method of dissolution can include centrifuging the tube containing a condensate formulation at the speed 12,000 g at 20° C.(or lower) temperature and then removing three quarters, 3/4V, of supernatant without disturbing the precipitated condensates at the bottom of the tube. Next a method can include adding 5× volume of a third reagent (e.g. reagent B1), V₃=5V, e.g., 1 mL, to the tube containing the condensate formulation. The method can further include mixing, which can be carried out by tapping the tube or repeated pipetting. Lastly, a method can include incubating the solution from the centrifuging and removal step at 37° C. for 15 minutes and mixing, by tapping the tube or repeated pipetting. The final solution volume 6V and measured final DNA/RNA concentration C_{XNA} can be recorded. The dissolved DNA or RNA samples can be stored at 4-8° C. for 1 week and used for the applications such as cell transfection or LNP synthesis.

According to some further embodiments of the present technology, thermostable DNA/RNA-LNP formulation kits or formulation buffer kits and methods of use are provided. In some instances, a kit can include one or more reagents, for instance a DNA/RNA-LNP formulation kit can include an LNP synthesis buffer, an LNP dialysis buffer, and an excipient mix. In one example, a DNA/RNA-LNP formulation reagent kit or buffer kit can include a (A1) LNP synthesis buffer, a (B1) LNP dialysis buffer, and a (B2) excipient mix. In some instances, a LNP synthesis buffer comprises an acid buffer consisting of at least one buffer including citrate or 1-2-3-4-butanetetracarboxylic acid at pH between 3 and 4 at buffer concentration 1 mM to 100 mM, and one or more excipients including phenylalanine arginine, glutamic acid, glycine, proline, tween, poloxamer, tween, and cyclodextrin at concentration from 0 to 500 mM. In some further instances, a LNP dialysis buffer comprises a formulation buffer consisting of at least one buffer including citrate (pH between 4.5 and 6.5) or 1-2-3-4-butanetetracarboxylic acid (pH between 4.5 and 6.5) or tris (pH between 6.0 and 8.5) or bis-Tris (pH between 6.0 and 8.5) at buffer concentration 1 mM to 100 mM. In some further instances, an excipient mix can comprise an excipient mix solution in the LNP dialysis buffer (e.g. buffer B1) containing one or more crowding agent including polyethylene glycol, sucrose, trehalose at concentration from 0 to 50%, by weight and one or more other excipients including phenylalanine arginine, glutamic acid, glycine, proline, tween, poloxamer, tween, and cyclodextrin at concentration from 0 to 500 mM.

In some further embodiments, methods of synthesizing DNA/RNA encapsulated LNPs are provided. In one example, with reference to the above described kit, a synthesis protocol can include a plurality of steps to prepare and synthesize DNA/RNA encapsulated LNPs. In a first step, a lipid mixture solution is prepared in absolute ethanol for LNP synthesis. The LNP lipids chosen typically comprise at least 4 components which can include a cationic or ionizable lipid (e.g., DLin-MC3-DMG), a helper lipid (e.g., DSPC), cholesterol, a PEG conjugated lipid (e.g., PEG2000-DMG). The lipid mixture solution should be prepared according to a user's LNP synthesis protocol. In a second step, a DNA or RNA solution is prepared in a first reagent, e.g. reagent A1 (LNP synthesis buffer). The above-described reagent kit provides multiple options of the first reagent (e.g. reagent A1) with different pH and buffer components as described in step 1. The specific pH and buffer components can be selected for a user's specific LNP lipid formulation and nucleic acids. The nucleic acid concentration is determined by the user's LNP synthesis protocol. In a third step, DNA/RNA-LNPs are synthesized by rapidly mixing the solutions of step 1 and step 2 and directing the products into the first reagent (e.g. reagent A1) of more than 20 times volume of the lipid solution of step 1. The LNP synthesis apparatus and the LNP synthesis process parameters, e.g., the total flow rate and aqueous to ethanol solution flow rate ratio can be based on a user's specific protocol. In a fourth step, buffer exchange is performed for the DNA/RNA-LNP products by two times of dialysis using a second reagent (e.g. reagent B1) at the volume ratio equal to 10 for each dialysis run. The dialysis apparatus and the process parameters can be based on a user's specific protocol. In a fifth step, the DNA/RNA-LNP products are concentrated after dialysis to about 1.25 times of the desired concentration of DNA/RNA-LNP through ultrafiltration. The ultrafiltration apparatus and the process parameters can be based on a user's specific protocol. In a sixth step, a third reagent (e.g. reagent B2) is added into the DNA/RNA-LNP product after ultrafiltration at the volume or weight ratio equal to 1 to 4. In the sixth step, mixing can be carried out by tapping the tube or repeated pipetting. This final DNA/RNA-LNP product can be aliquoted and stored at ambient temperature (e.g. ˜20)° C. or in a refrigerator at 4-8 ºC for at least a month. The maximum shelf life depends on the specific DNA/RNA-LNP products. With respect to the synthesis protocol, it will be appreciated that the volume or weight ratio of the third reagent (e.g. reagent B2) and DNA/RNA-LNP in the sixth step is an example. Other ratios can also be used with the concentrations of reagents adjusted accordingly. Further, the final DNA/RNA-LNP products from step 6 can also be stored in freezers at up to −20° C. or −80° C. However, freeze-thaw cycles for these DNA/RNA-LNP products should be minimized and at no more than three.

### EXAMPLES

In vitro RNA condensate formation in citrate buffer.

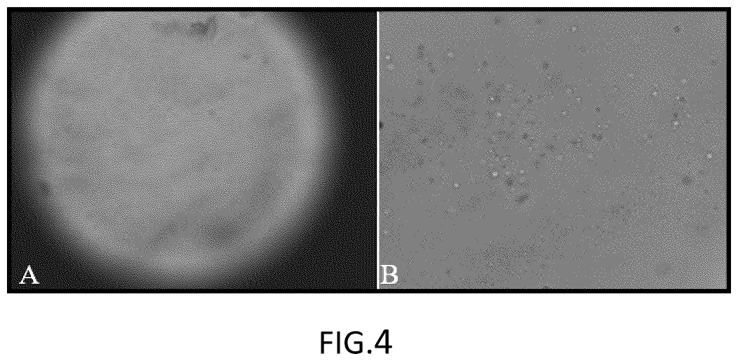

In vitro RNA condensates were first produced through LLPS in 10 mM pH 6 citrate buffer (the common manufacture buffer of commercial mRNA). To induce LLPS in RNA solutions, high salt concentration (500 mM NaCl) was used to reduce the repulsion between the negatively charged RNA molecules and a macromolecular crowding reagent, PEG-3350, was used to increase the attractive interactions between RNAs. As illustrated in FIG. 4, droplet-like condensates were observed in polyA RNA solutions with 500 μg/mL poly A, 500 mM NaCl, 2 μL and various PEG concentrations from 6%-12% (w/w). FIG. 4A shows 8% PEG 5 mM citrate at 4× magnification and FIG. 4B shows 8% PEG 5 mM citrate at 20× magnification. According to various embodiment of the present technology, varying concentrations and molecular weights of macromolecular crowding reagent can be used, for instance PEG having a molecular weight from about 1,000 to about 20,000 Da and in the concentration range from about 0% to about 60% (w/w).

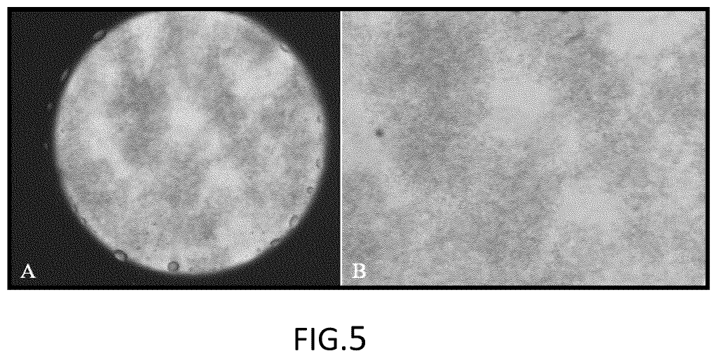

Subsequent to the Poly-A RNA condensate formation, an mRNA (e.g. Dash GFP mRNA, Aldevron) was utilized to demonstrate that similar RNA condensates can be made for mRNAs that are long RNAs, often with extensive secondary and tertiary structures. Referring to FIG. 5, the solution conditions are: 10 mM citrate buffer 0.5 M NaCl pH 6, 10% (w/w) PEG, and 500 ug/mL Dasher GFP. FIG. 5A shows GFP at 4× magnification and FIG. 5B shows GFP at 20× magnification.

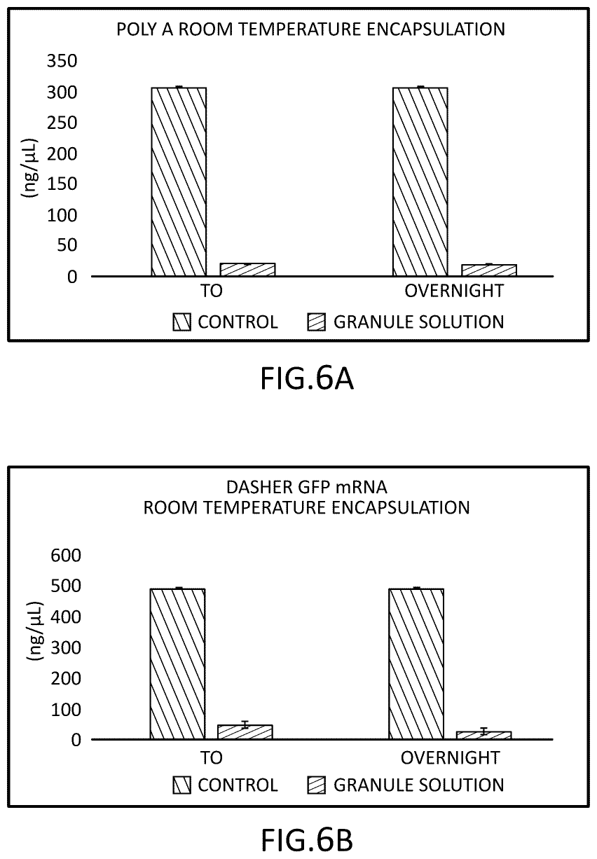

In order to make useful RNA formulations, a high percentage of RNA needs to be encapsulated in the condensates. Therefore, in accordance with the present technology and with reference to FIG. 6, >95% RNA molecules can be encapsulated in the condensates. The following solution was prepared: 10 mM buffer (PBS pH 7.4 or citrate pH 6) 0.5 M NaCl, 10% (w/w) PEG-3350, and 500 μg/mL RNA. A control was also prepared that replaced PEG with RNase-free water. Half of the solutions were incubated for one hour before the first measurement is taken, and the other half were incubated overnight. After an hour incubation at a given temperature, the solutions were centrifuged for three minutes at 4000 relative centrifugal force (rcf), and the concentration of the RNA in the supernatant measured using a Nanodrop spectrophotometer. 2 μL of the supernatant was carefully pipetted onto the Nanodrop. As illustrated in FIG. 6, concentrations of Poly-A and the GFP mRNA in supernatants after encapsulation in the condensates at room temperature. The data for the condensate formulation and the condensate-free control solution are shown in blue and orange, respectively.

Dissolution of RNA condensates.

Usefulness of the RNA condensates as a pharmaceutical formulation requires them to be dissolvable upon reconstitution or heating before administration. The droplet-like morphology of the RNA condensates is consistent with LLPS, a phase separation reversible upon dilution and/or heating. To confirm that the RNA condensates were formed through LLPS, the reversibility of these condensates were studied. Condensate formation could be reversed by either increasing the temperature of the solution or diluting the solution to decrease the concentration of RNA. First, solutions from the RNA condensate formation with varying PEG were diluted. 6% and 8% PEG solutions were chosen as they both resulted in condensate formation. Once the solutions were diluted two times and incubated at room temperature for fifteen minutes, they were imaged. Any condensates that formed before dilution were shown to have dissipated.

Another illustrative method illustrating that condensate formation is reversible according to the present technology, is by increasing temperature. This solution was taken from the experiments incorporating varying PEG concentrations. Once the microscope well was incubated at room temperature for 15 minutes, it was imaged to show condensates had formed. Then the well was incubated at 37ºC for 15 more minutes and reimaged, which showed that the droplets had dissipated.

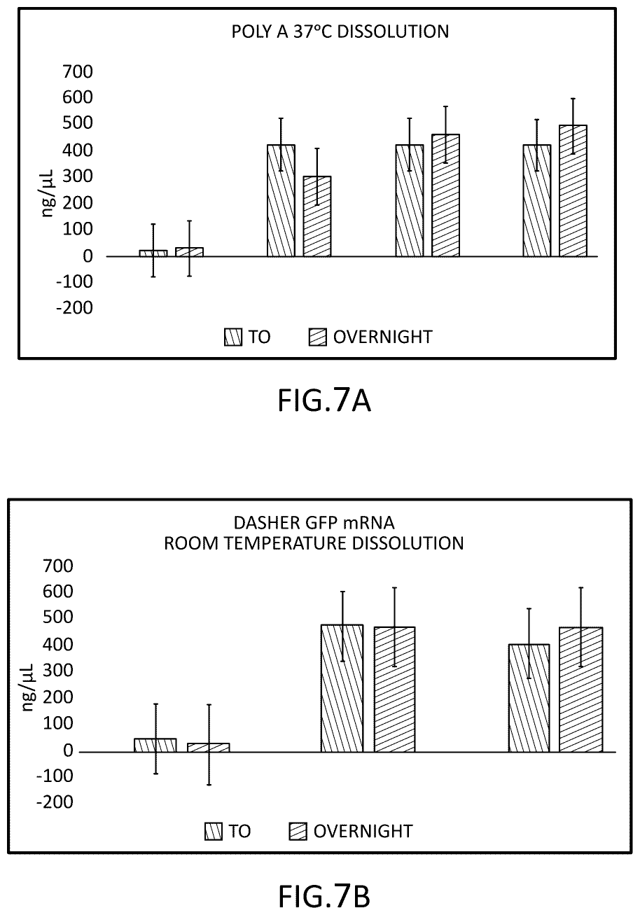

Referring to FIG. 7, it is illustrated that after being diluted two times the RNA in the condensates completely dissolve back into solution. The solution conditions are: 10 mM citrate buffer 0.5 M NaCl pH 6, 10% (w/w) PEG, and 500 ng/uL RNA. As illustrated, FIG. 7 shows dissolution of RNA condensates upon 2× dilution, e.g. with water. The left bars indicate RNA concentration in the supernatant over condensates before dilution. The rest of the bars to the right indicate RNA concentration after dissolving the condensates by dilution. RNA concentrations were corrected by the dilution factors to show the concentration in the original solution volume before dilution. FIG. 7A shows the dissolution of Poly-A in a condensate after diluted with water 2 times and incubated at 37° C. for 15 minutes (the RNA concentrations were adjusted by the dilution factor, and FIG. 7B shows the dissolution of GFP mRNA in a condensate after diluted with water 6 times and incubated at 37° C. for 15 minutes (the RNA concentrations were adjusted by the dilution factor).

RNA condensate formation in PBS buffer.

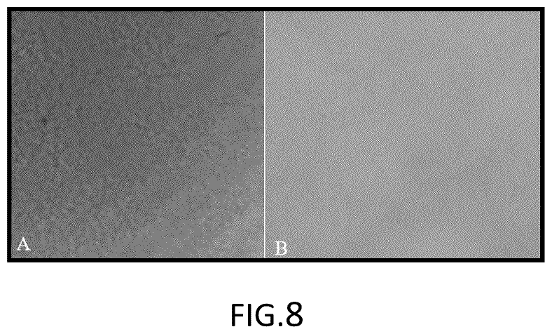

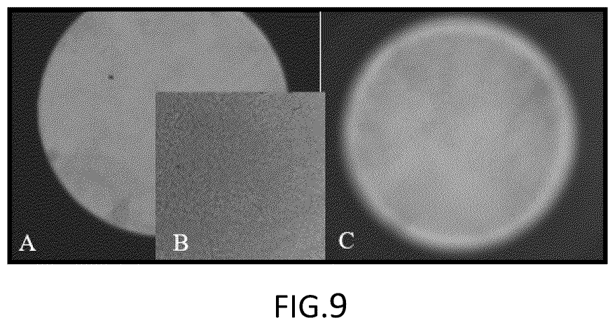

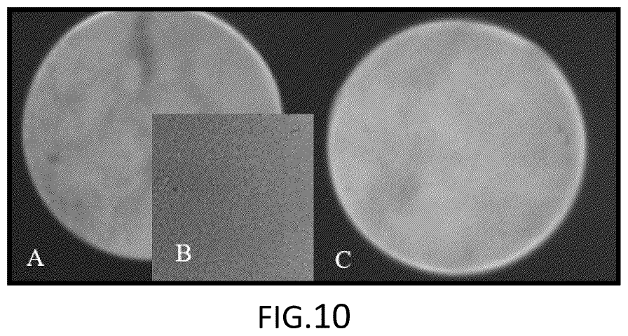

After demonstrating RNA condensates formation in citrate buffer, it is also shown that such condensate formation can be induced in other buffers. Phosphate buffered saline (PBS) was chosen for this example. With reference to FIGS. 8-10, first, condensate formation with varying PEG % was explored to decide what percent would work best with PBS buffer at 21° C. and and 5° C. The solution conditions used were: 10 mM PBS buffer 0.5 M salt pH 7.4, 500 ng/ml Dasher GFP mRNA, and the PEG concentration was varied from 4-12%. FIG. 8A shows 6% PEG, RT, 20× magnification with phase contrast; and FIG. 8B shows 6% PEG, 5° C., 20× magnification. FIG. 9A shows 8% PEG, RT at 4× magnification, FIG. 9B shows 8% PEG, RT at 20× magnification, and FIG. 9C shows 8% PEG, 5° C. at 4× magnification. FIG. 10A shows 10% PEG, RT, at 4× magnification, FIG. 10B shows 10% PEG, RT, at 20× magnification, and FIG. 10C shows 10% PEG, 5° C., at 4× magnification.

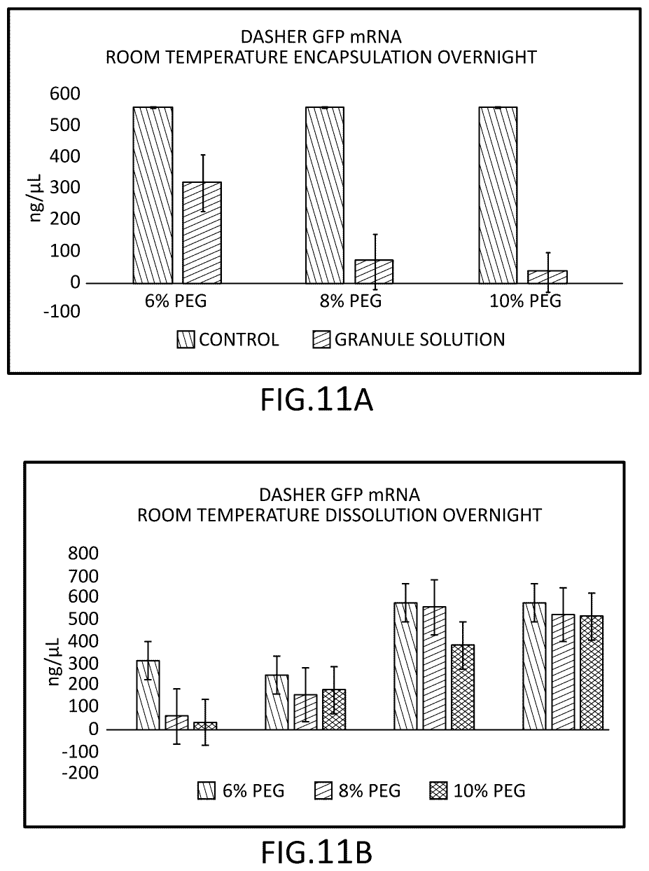

The RNA encapsulation study conducted earlier with PolyA and Dasher GFP in citrate buffer were repeated for the PBS buffer pH 7.4 conditions. With reference to FIG. 11, the encapsulation of solutions made with 6%, 8%, and 10% PEG were also compared in PBS buffer. FIG. 11A shows concentrations of GFP mRNA in supernatants after encapsulation in the condensates at room temperature with various PEG concentration (6%, 8%, and 10%). The data for the condensate formulation and the condensate-free control solution are shown in blue and orange, respectively. FIG. 11B shows dissolution of the RNA condensates upon 2× dilution. The left bars indicate RNA concentration in the supernatant over condensates before dilution. The rest of the bars to the right indicate RNA concentration after dissolving the condensates by dilution. RNA concentrations were corrected by the dilution factors to show the concentration in the original solution volume before dilution.

Effect of solution salinity on RNA condensate formation.

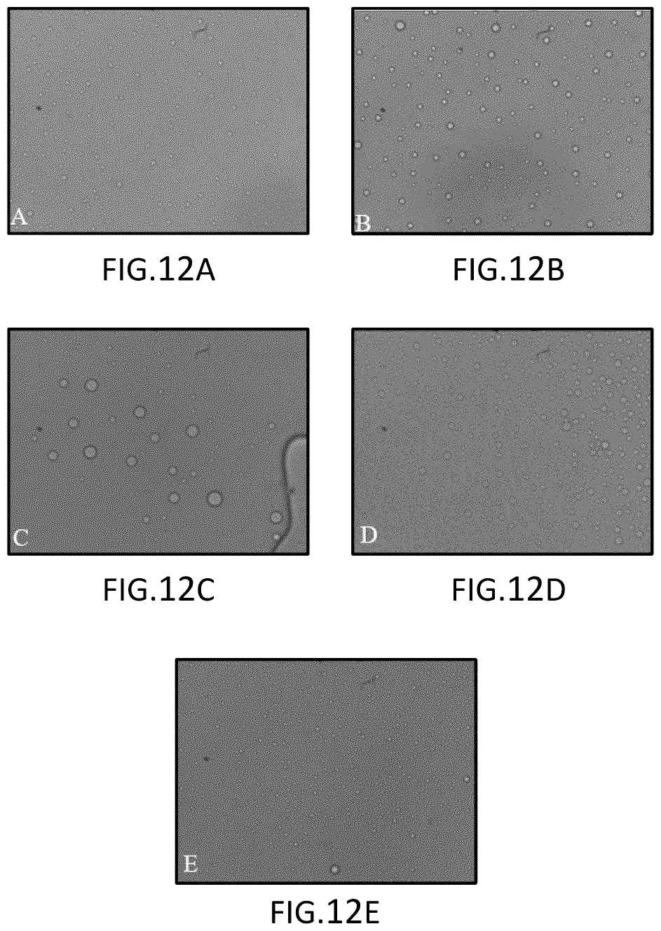

As will be appreciated, for pharmaceutical formulations it is important that RNA condensates can be prepared at various salinity of solutions. Accordingly, the RNA condensate formation under different salinity conditions were investigated. With reference to FIG. 12, RNA condensates were observed in 10 mM pH 6 citrate buffer with 10% (w/w) PEG, 500 μg/mL polyA RNA, and various NaCl concentrations from 0.18 to 0.58 M. The solutions were prepared and incubated for 15 minutes in the microscope wells before viewing. Sodium chloride solutions of known concentration were run through an HPLC machine to measure conductivity. FIG. 12A. shows 0.18 M NaCl, at 20× magnification; FIG. 12B. shows 0.35 M NaCl, at 20× magnification; FIG. 12C shows 0.40 M NaCl, at 20× magnification; FIG. 12D shows 0.5 M NaCl, at 20× magnification; and FIG. 12E shows 0.58 M NaCl, at 20× magnification.

RNA condensate formation in the presence of cationic polymers.

To be of use in methods for preparing pharmaceutical grade formulations, it is important to be able to be able to induce RNA condensate formation by LLPS at a broad range of solution conditions (including temperature, buffer conditions, RNA concentration, and other excipient concentrations). To make a versatile method for preparing RNA condensate formulations, it is demonstrated that RNA condensates can be induced by addition of cationic polymers.

Various cationic proteins were found in natural P-body condensates. It is postulated that these positively charged proteins could neutralize the negative charges on RNAs and thereby facilitate condensate formation by reducing the net repulsion. To test this electrostatic effect, condensate formation of poly-A has been investigated in the presence of cationic polymers. These cationic polymers will lack secondary structure, similarly to the IDR regions of proteins, and will have positively charged components that will be attracted to the negatively charged phosphate backbone of the RNA. These cationic polymers were also chosen because of their function in plasmid DNA transfection or lack of toxicity to mammalian cells. Polyethylenimine (PEI), for example, has been used in mammaliancell transfection of plasmids. Other cationic polymers have been approved by the FDA for use in pharmaceutical drugs. Since the cationic polymers will be used in mammalian cell transfection experiments at a later time, the initial search on cationic polymers should focus on polymers that will not contribute to cell death. The concentrations of cationic polymers were determined to achieve the Nitrogen-Phosphorus ratio, N/P: 1 and N/P: 6.

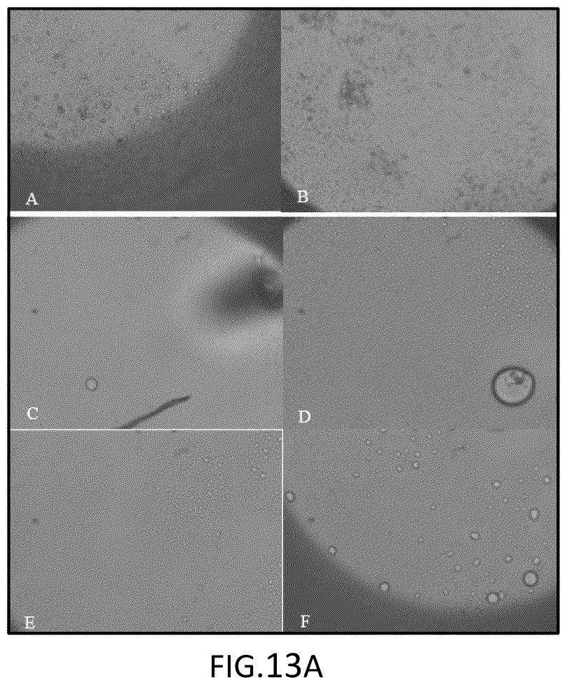

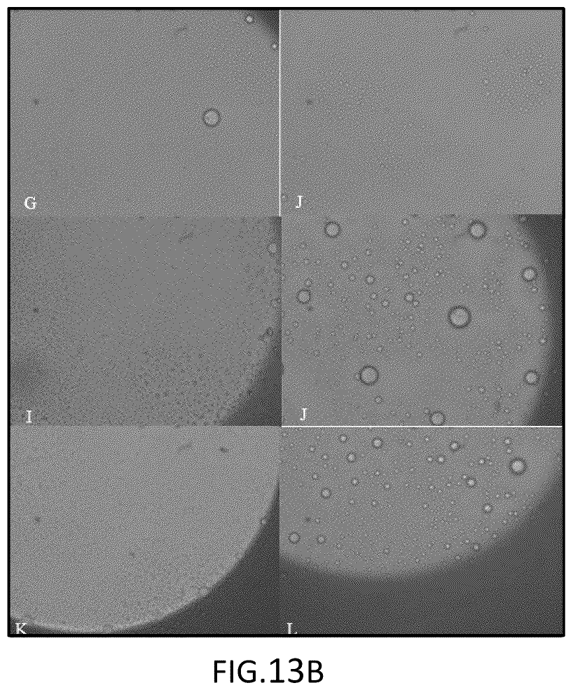

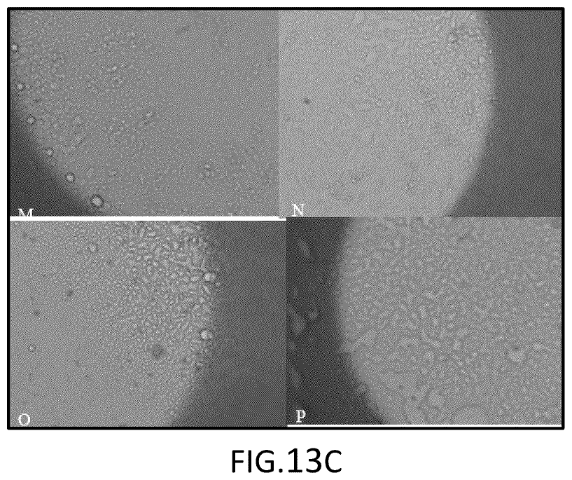

The following were combined in a microscope well to look for condensate formation: 10 mM citrate buffer pH 6 0.5 M NaCl, 10% (w/w) PEG, 500 μg/mL PolyA RNA, and N/P: 1 or N/P: 6 cationic polymer. The control replaced PolyA RNA with water to determine if the cationic polymer phase separated on its own. The solutions were incubated at room temperature for 15 minutes before imaging. Every cationic polymer formed a condensate with PolyA, and two cationic polymers, PVP COVA 13k N/P: 6 and PDMAEMA N/P: 6, phase separated without the presence of RNA (See FIG. 13. All images were taken at 20× magnification). FIGS. 13A-13C illustrate RNA condensate formation in the presence of cationic polymers. FIG. 13A shows a) Polyethyleneimine 10k N:P=6; b) PEI 10k, N:P=1; c) polyvinylpyrrolidone 3.5k N:P=6; d) PVP 3.5, N:P=1; e) PVP 8k, N:P=6; f) PVP 8k, N:P=1. FIG. 13B shows g) PVP 40k, N:P=6; h) PVP 40k, N:P=1; i) Poly(l-vinylpyrrolidone-co-vinyl acetate) (PVPCOVA) 13k, N:P ratio=6; j) PVPCOVA 13k, N:P ratio=1; k). PVPCOVA 50k, N:P ratio=6; 1) PVPCOVA 50k, N:P ratio=1. FIG. 13C shows m) Poly[2 (Dimethylamino)ethyl Methacrylate] (PDMAEMA), N:P ratio=6; n) PDMAEMA, N:P ratio=1; o). Protamine Sulfate, N:P ratio=6; p) Protamine Sulfate, N:P ratio=1.

mRNA degradation in the in vitro condensates in citrate buffer at pH 6.

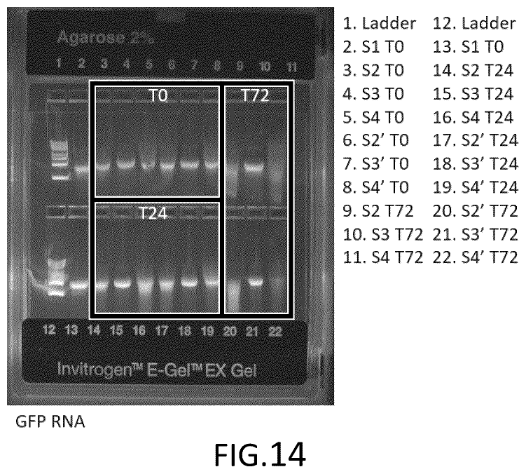

According to aspects of the technology described herein, it is demonstrated that the condensates protect RNA against degradation. Solutions were prepared according to materials and methods for the RNA degradation experiment for the GFP mRNA. As shown in FIG. 14, starting 24 hours after incubation at room temperature, solutions without PEG and solutions without salt that would not form condensates began to show degradation. Solutions that would form condensates did not show significant degradation until the 72-hour incubation time. As illustrated FIG. 14 shows an agarose gel test of GFP mRNA degradation in samples: S1, fresh mRNA control (no condensates); S2, mRNA with salt (no condensates); S3, mRNA in condensates; S4, mRNA without salt with PEG (no condensates). Each timepoint has three samples run in duplicate.

In an embodiment, a method for preparing a stable nucleic acid liquid formulation comprises providing an RNA solution having an initial RNA concentration, adding at least one polymer, adding at least one salt, and forming one or more macroscopic RNA-rich condensates, which in some instances can have various morphologies including granules, droplets, and amorphous aggregates.

In an embodiment, a method for preparing a stable nucleic acid encapsulated lipid nanoparticles (DNA/RNA-LNP) liquid formulation is provided. In some aspects, a method comprises providing a DNA or RNA solution in an LNP synthesis buffer containing at least one nucleic acid stabilizing buffer salt and at least one other nucleic acid stabilizing excipient, the DNA or RNA having an initial concentration, synthesizing DNA or RNA encapsulated LNPs based on the DNA or RNA solution and the LNP synthesis buffer, wherein the LNP synthesis buffer is used as the quenching buffer, preparing a plurality of formulation product buffers containing at least one nucleic acid stabilizing buffer salt and at least one other nucleic acid stabilizing excipient, wherein the formulation product buffers have a series of concentrations, performing buffer exchange for the DNA or RNA encapsulated LNPs using a LNP product formulation buffer without a macromolecular crowding reagent, adding a macromolecular crowding reagent and at least one nucleic acid stabilizing excipient at the series of concentrations to the DNA/LNP-LNP formulation after buffer exchange to achieve osmotic pressure and chemical potentials close-to-equal to the LNP synthesis buffer, and forming the stable nucleic acid encapsulated lipid nanoparticle liquid formulation. In some aspects, the (e.g. RNSA) stabilizing buffer for LNP synthesis buffer (i.e. LNP synthesis buffer) comprises citrate or 1-2-3-4-butanetetracarboxylic acid, at pH between 3 and 4, and in some instances, cannot or does not contain acetate. In some other aspects, the stabilizing buffer (e.g. RNA stabilizing buffer) for LNP product formulation buffer are tris, bis-Tris, citrate, or 1-2-3-4-butanetetracarboxylic acid at pH between 6 and 8.5. In some aspects, the other nucleic acid stabilizing excipient comprises at least one of phenylalanine arginine, glutamic acid, glycine, proline, tween, poloxamer, tween, and cyclodextrin. In some further aspects, the macromolecular crowding agent is polyethylene glycol.

Embodiments described herein can be understood more readily by reference to the examples described above. Elements, apparatus, and methods described herein, however, are not limited to any specific embodiment presented in the Examples. It should be recognized that these are merely illustrative of some principles of this disclosure, and are non-limiting. Numerous modifications and adaptations will be readily apparent without departing from the spirit and scope of the disclosure.

Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below. Certain features and subcombinations are of utility and can be employed without reference to other features and subcombinations and are contemplated within the scope of the claims.

1. A method for preparing a stable nucleic acid liquid formulation comprising:
providing a DNA or RNA solution having an initial DNA or RNA concentration;
adding at least one polymer;
adding at least one salt; and
forming a macroscopic DNA-rich or RNA-rich condensate.

2. The method of claim 1, wherein the DNA or RNA solution is in at least one of a nucleic acid stabilizing buffer and one or more other nucleic acid stabilizing excipients.

3. The method of claim 2, wherein the nucleic acid stabilizing buffer is at least one of citrate, 1-2-3-4-butanetetracarboxylic acid, tris, bis-Tris, citrate, and 1-2-3-4-butanetetracarboxylic acid.

4. The method of claim 2, wherein the one or more other nucleic acid stabilizing excipients comprise at least one of phenylalanine arginine, glutamic acid, glycine, proline, tween, poloxamer, tween, and cyclodextrin.

5. The method of claim 1, wherein the polymer is polyethylene glycol (PEG).

6. The method of claim 1, wherein the salt is sodium chloride (NaCl).

7. The method of claim 1, wherein the RNA solution is an mRNA solution.

8. The method of claim 1, wherein greater than 95% of the DNA or RNA in solution is partitioned into the DNA-rich or RNA-rich condensate.

9. The method of claim 1, wherein the DNA-rich or RNA-rich condensate is separated by centrifugation.

10. The method of claim 1 further comprising: dissolving the DNA-rich or RNA-rich condensate in water and other diluents.

11. The method of claim 10, wherein the DNA or RNA concentration is restored to the initial DNA or RNA concentration.

12. The method of claim 1, wherein there is no significant degradation of DNA or RNA in the DNA-rich or RNA-rich condensate for at least three days at ambient temperature.

13. The method of claim 1, wherein the polymer is at least one of a biocompatible crowding polymer and a cationic polymer.

14. The method of claim 10, wherein the cationic polymer is polyvinyl pyrrolidone.

15. A composition comprising a DNA-rich or RNA-rich condensate formed by the method of claim 1.

16. A kit for thermostable DNA/RNA condensate formulation, the kit comprising:
a buffer;
a polymer salt mix; and
a diluent.

17. The kit of claim 13, wherein the buffer comprises at least one of citrate, 1-2-3-4-butanetetracarboxylic acid, tris, and bis-tris, and one or more excipients comprising at least one of phenylalanine arginine, glutamic acid, glycine, proline, tween, poloxamer, tween, and cyclodextrin.

18. The kit of claim 13, wherein the polymer salt mix comprises a salt and a polymer.

19. The kit of claim 13, wherein the diluent comprises at least one of nuclease-free water, citrate, and tris.

20. A kit for thermostable DNA/RNA-LNP formulation, the kit comprising:
a LNP synthesis buffer;
a LNP dialysis buffer; and
an excipient mix.

21. The kit of claim 17, wherein the LNP synthesis buffer comprises an acid buffer and one or more excipients.

22. The kit of claim 17, wherein the LNP dialysis buffer comprises at least one of citrate, 1-2-3-4-butanetetracarboxylic acid, tris, and bis-tris.

23. The kit of claim 17, wherein the excipient mix comprises at least one of polyethylene glycol, sucrose, and trehalose, and one or more of phenylalanine arginine, glutamic acid, glycine, proline, tween, poloxamer, tween, and cyclodextrin.

24. A method method for preparing a stable nucleic acid encapsulated lipid nanoparticles (DNA/RNA-LNP) liquid formulation comprising:
providing a DNA or RNA solution in an LNP synthesis buffer containing at least one nucleic acid stabilizing buffer salt and at least one other nucleic acid stabilizing excipient, the DNA or RNA having an initial concentration;
synthesizing DNA or RNA encapsulated LNPs based on the DNA or RNA solution and the LNP synthesis buffer, wherein the LNP synthesis buffer is used as the quenching buffer;
preparing a plurality of formulation product buffers containing at least one nucleic acid stabilizing buffer salt and at least one other nucleic acid stabilizing excipient, wherein the formulation product buffers have a series of concentrations;
performing buffer exchange for the DNA or RNA encapsulated LNPs using a LNP product formulation buffer without a macromolecular crowding reagent;
adding a macromolecular crowding agent and at least one nucleic acid stabilizing excipient at the series of concentrations to the DNA/LNP-LNP formulation after buffer exchange; and
forming the stable nucleic acid encapsulated lipid nanoparticle liquid formulation.

25. The method of claim 24, wherein the LNP synthesis buffer are citrate or 1-2-3-4-butanetetracarboxylic acid, at pH between 3 and 4 (and cannot contain acetate).

26. The method of claim 24, wherein the LNP product formulation buffer comprises at least one of tris, bis-Tris, citrate, or 1-2-3-4-butanetetracarboxylic acid at pH between 6 and 8.5.

27. The method of claim 24, wherein the other nucleic acid stabilizing excipient comprises at least one of phenylalanine arginine, glutamic acid, glycine, proline, tween, poloxamer, tween, and cyclodextrin.

28. The method of claim 24, wherein the macromolecular crowding reagent is polyethylene glycol.

29. A composition comprising a DNA or RNA encapsulated lipid nanoparticle solution formed by the method of claim 24.
